(19) United States
(12) Patent Application Publication
(10) Pub. No.: US 2017/0299289 A1
(43) Pub. Date: Oct. 19, 2017

(54) UV DECONTAMINATION SYSTEM FOR CLIMATE CONTROL SYSTEMS

(71) Applicant: SANUVOX TECHNOLOGIES INC., Montreal (CA)

(72) Inventors: Normand BRAIS, Rosemere (CA); Benoit DESPATIS PAQUETTE, Laval (CA); Jocelyn DAME, Saint-Lazare (CA)

(21) Appl. No.: 15/634,618

(22) Filed: Jun. 27, 2017

Related U.S. Application Data
(63) Continuation of application No. PCT/CA2016/050022, filed on Jan. 11, 2016.
(60) Provisional application No. 62/102,019, filed on Jan. 10, 2015.

(51) Int. Cl. F28G 13/00; F24F 13/30; A61L 2/10; A61L 2/26

(52) U.S. Cl. CPC F28G 13/00; A61L 2/26; F24F 13/30; A61L 2/10; F24F 2221/22; A61L 2202/11

(57) ABSTRACT

Disclosed is a decontamination system for climate control systems such as air conditioning systems and in particular a UV decontamination system for cooling coils. The UV decontamination system is suited for installing downstream from the cooling coil and has anti-fouling protection, anti-cooling UV lamp optimization, and angular optimization for maximum penetration into the cooling coil, inter alia. The UV decontamination system may comprise a reflector a deflector and a UV lamp and other components. This abstract is not to be considered limiting.

This application is a continuation of patent application PCT/CA2016/050022 filed Jan. 11, 2016, that claims priority of U.S. provisional patent application 62/102,019 filed Jan. 10, 2015.

### TECHNICAL FIELD

The subject matter disclosed relates generally to the field of air conditioning and sanitization of air conditioning units and sanitization of cooling coils. In particular, the subject matter relates to UV sanitization for air conditioning units, and more particularly cooling coils.

### BACKGROUND

Cooling coils are used in climate control systems such as air conditioning systems. Typically, a cooling coil comprises a tubular body, e.g. piping that travels a certain distance in thermal contact with an external fluid from which it absorbs heat. Inside the cooling coil, an internal fluid near its evaporation point is heated by the external fluid and typically undergoes evaporation in the cooling coil, thereby cooling the coil and the external fluid. The internal fluid typically is compressed at the egress of the cooling coil and transferred to a secondary coil where it typically undergoes condensation before being depressurized and re-fed into the cooling coil.

In particular, in an air conditioning system, a cooling coil is typically placed in the path of forced air, which comes into contact with the coil to heat the coil and simultaneously cool the air. Condensation typically forms on the cooling coil which can be carried out as mist by the forced air. Moreover the cooling coil forms conditions that can be as unhealthy for building occupants as they are unpleasant. Unhygienic coil conditions can lead to bad odors and while the smell associated with mold growth is a bad situation, mold growth on coils also has a detrimental effect on system efficiency. This degradation of performance ultimately leads to higher energy costs.

Typically, there are four main conditions will result in mold and fungus biofilm growth:
1. A source of mold spores. Sufficient mold spores are found in nearly every environment and brought into the building through door openings and outdoor air supplies.
2. Organic material on which the mold can grow. Dust and particles of organic material are also readily available in every system, even with the best filtration systems.
3. The right temperature range. Temperatures from 15° C. (60° F.) to 40° C. (104° F.) provide the adequate incubation range.
4. Moisture, which is in more than adequate supply on cooling coils and drain pans of all air conditioning units.

The odors associated with this problem stem from the natural growth and decay of mold. As the molds break down they decay into toxins and volatile organic compounds (VOCs), which cause the characteristic odor. In addition, some people have allergic reactions to these toxins and VOCs. These problems certainly contribute to poor building air quality, but mold growth does also impact energy consumption through lost heat transfer efficiency as well.

A typical cooling coil will be comprised of parallel densely-packed thin metal fins which provide wide surface area for heat exchange. The space between such fins can be quite narrow, resulting in blockage which impacts airflow across the cooling coil.

Over the years, numerous mechanical/chemical coil-cleaning methods have been used to control this problem. Some of those techniques involve the use of high pressure washer with detergents, acids, and solvents, which can pose health and safety issues as well as diminish the life of the coil. Often coil cleaning isn't done with regularity and even when it is done on schedule, the mold growth can recolonize the coil within a very short time given the exponential growth observed generally.

However, recent evidence suggests that these methods are too often ineffective. Chemical cleaning may only remove surface growth while leaving material still embedded in the center of the fin pack. Some reports indicate steam cleaning can actually force the surface growth deeper in the fin pack compressing the growth material so tightly that the only solution may be a new coil. Both methods can also be detrimental to some of today's enhanced corrugated metal coil surfaces. Coil cleaning cannot be done in practice with the frequency and level that would keep the coil operating at design conditions on a daily basis.

### SUMMARY

In accordance with a broad embodiment, there is provided a cooling unit for a climate control system. The unit comprises a finned cooling coil comprising a fluid conductor occupying a coil volume having an upstream side and a downstream side, the finned cooling coil including a finned fluid passage between the upstream side and the downstream side allowing flow of air through the finned cooling coil between the upstream side and the downstream side. The unit further comprise an ultraviolet irradiation unit mounted on the downstream side of the coil volume for irradiating the finned cooling coil with ultraviolet light for decontaminating the finned cooling coil. The ultraviolet irradiation unit comprises an ultraviolet lamp having an electrical power source for powering an ultraviolet light source and a lamp mount for accommodating the ultraviolet light source in a lamp volume. The ultraviolet irradiation unit further comprises a reflector mounted downstream from the lamp volume and having a ultraviolet light-reflective upstream surface oriented generally upstream to reflect ultraviolet radiation from the ultraviolet light source towards the finned cooling coil. The ultraviolet irradiation unit further comprises a deflector mounted upstream from the lamp volume creating an obstruction deflecting air flow away from the lamp volume.

In accordance with another broad embodiment, there is provided a ultraviolet decontamination unit for decontaminating a finned cooling coil in a climate control system comprising: a first side for facing the finned cooling coil, a second side opposite the first side and a body defined between the front and rear side and mountable in the climate control system in proximity to a finned cooling coil with an orientation wherein the front side faces the finned cooling coil and an airflow in the climate control system flows generally towards the front side of the body. The body comprises an ultraviolet lamp located between the front and rear side for generating ultraviolet light to irradiate the finned cooling coil having an electrical power source for powering an ultraviolet light source and a lamp volume for accommodating the ultraviolet light source. The body further comprises a reflector located towards the rear from the lamp volume and having a first ultraviolet light-reflective front face oriented towards the lamp volume to reflect ultraviolet radiation from the ultraviolet light source towards the front. The body further comprises a deflector located towards the front from the lamp volume creating an obstruction and having a geometry for deflecting the air flow away from the lamp volume to limit cooling of the ultraviolet light source.

### BRIEF DESCRIPTION OF THE DRAWINGS

The invention will be better understood by way of the following detailed description of embodiments of the invention with reference to the appended drawings, in which:

FIG. 1 shows a front perspective view of a cooling coil in a climate control system in accordance with a particular example;

FIG. 2 shows a perspective view of a UV decontamination system installed in the climate control system of FIG. 1;

FIG. 3 shows a cross-section of the cooling coil of FIG. 1 taken about line A-A;

FIG. 4 shows a top-down cross-sectional view a UV decontamination system in accordance with another particular example;

FIG. 5 shows a top-down cross-sectional view a UV decontamination system in accordance with another particular example;

FIG. 6 shows a top-down cross-sectional view a UV decontamination system in accordance with another particular example;

FIG. 7 shows a top-down cross-sectional view of a cooling coil;

FIG. 8 shows a top-down cross-sectional view a UV decontamination system in accordance with another particular example;

FIG. 9 shows a side elevation view of a ballast according to a particular example;

FIG. 10 shows a top-down cross-sectional view a UV decontamination system in accordance with another particular example; and

FIG. 11 shows a perspective view of a cooling coil with fins present with an enlarged portion showing details.

### DETAILED DESCRIPTION

Using germicidal UVC coil cleaning by irradiation, in essence, with UVC lights, coil cleaning becomes a continuous, non-contact automatic and labor-free alternative. The UV-C light works by attacking the DNA of the mold and rendering it sterile so that it cannot reproduce. Contrasting physical cleaning methods to the use UV-C light is analogous to the difference between treating the symptoms versus curing the disease. UVC technology is not new, as it has proven itself for over 75 years as a way to provide sterilization of drinking water. It has also been used for medical and food processing applications.

The effectiveness of the UVC light is a function of the light intensity in watt per square meter and the exposure time in seconds. Aluminum coil fins are a good reflective surface and, as a result, the UVC energy is capable of penetrating three- and four-row coils with excellent results. Given continuous exposure, UV-C lights can clean up a coil already contaminated by mold growth and keep the coil cleaner than other methods.

The application of germicidal ultraviolet light in air-handling systems now allows for a proactive method for keeping the coil clean and operating in “as new” performance all the time. UVC lights can be added to air handlers and other pieces of equipment through a relatively simple retrofit kit, and nowadays many OEM manufacturers also offer them as a factory-installed option in HVAC equipment. Interestingly, while UV-C light has been promoted for its positive impact on indoor air quality (IAQ), the “bottom line” impact—its contribution to system efficiency and lower maintenance costs—might ultimately be considered to be its greatest asset.

If properly sized and designed, UVC light systems provide an efficient proactive coil cleaning method that can keep coils operating at near design conditions year round. Coils kept free of a bio-film perform far better, saving energy and avoiding the odors associated with a locker room. Reducing energy costs and improving air quality for building occupants creates the ideal scenario for any building owner or manager.

Given the limited available space, the UVC light ends up being positioned about a foot or two from the coil surface and, in most cases, this available space is found to be downstream of the coil. According to the “ASHRAE Handbook, HVAC Systems and Equipment, Chapter 16”, entitled “Ultraviolet Lamp Systems”, UVGI Systems can be installed Upstream or Downstream of the Cooling Coil. Both locations have advantages and disadvantages. A problem arises when the UV-C light system is installed downstream of the cooling coil, where the lamps are exposed to condensing water mist carried by the cold air stream. The UVC light systems may then loose well over 50% of their effectiveness over short periods of time due to the impact of cold water mist hitting the lamp and leaving behind a fouling film residue after evaporation.

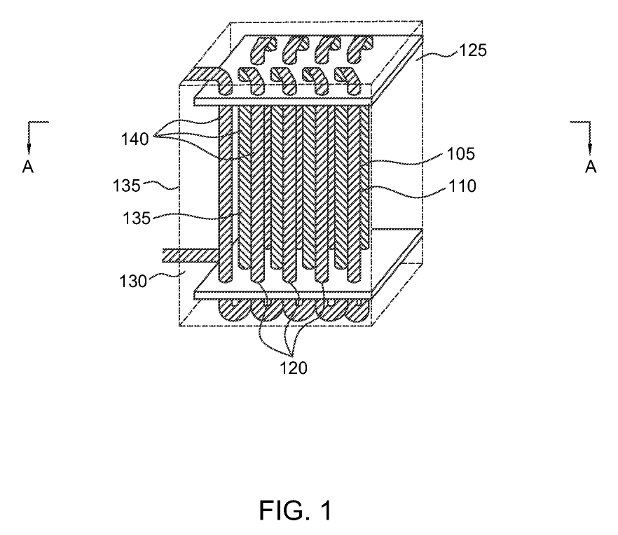

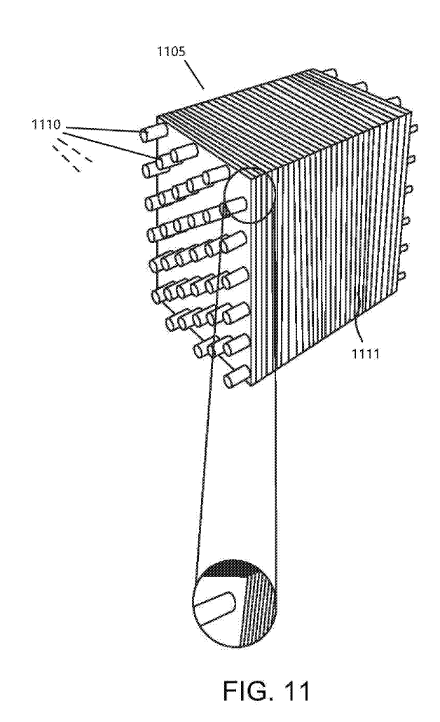

FIG. 1 shows a cooling coil 105 in a climate control system. The coil 105 occupies a coil volume 115 which is in a climate control system. The coil 105 comprises finned tubing 110 that carries cold fluid. For the purposes of illustrating the tubing 110, the fins are not shown here. FIG. 11 shows a cooling coil 1105 with the ends of a tubing 1110 cut-away, but showing fins 1111. Typically, fins 1111 may be 5.5 thousandth of an inch (0.14 mm) wide and may be spaced apart width-wise 6-18 fins per linear inch (2.54 cm). As shown in FIG. 11 and even more visible in the enlarged portion, the fins are arranged parallel to one another.

In order to cool the air, the finned tubing 110 is put in contact with air inside the climate control system in order to cool it. To this end, the climate control system comprises a source of air flow, e.g. a fan or a duct carrying forced air creating an air flow directed to the finned coil volume 115 and more particularly to the coil 105. In order to expose the finned tubing 110 to the air and to allow airflow, the coil 105 comprises a fluid passage, particularly here an air passage 120, through which the air can flow and be exposed to and in contact with the finned tubing 110.

In this example, the finned tubing 110 is continuous, however in certain systems there could be multiple closed circuits and therefore multiple coils arranged together as one within the coil volume 115.

The cooling coil 105 is typically mounted within an air path circuit that may be enclosed, e.g. partially, within ducting to force air flow through the air passage 120. The coil volume 115 therefore has an upstream side 125 and a downstream side 130. At the upstream side 125 is the upstream facing portion of the coil 105 and at the downstream side 130 is the downstream facing portion of the coil 105.

The fluid passage is provided by finned spaces between portions of the coil 105 that form one or more path from the upstream side 125 to the downstream side. Typically, the fluid passage is interrupted by many portions of the coil tubing 105, since the coil will typically be bent so as to have a greater length or surface area within the coil volume 115. Thus the fluid passage may comprise many different paths from the upstream side to the downstream side. In the example shown here, the space between spaced-apart straight portions 140 of the coil 105, and between the fins provided there, provide the air passage 120.

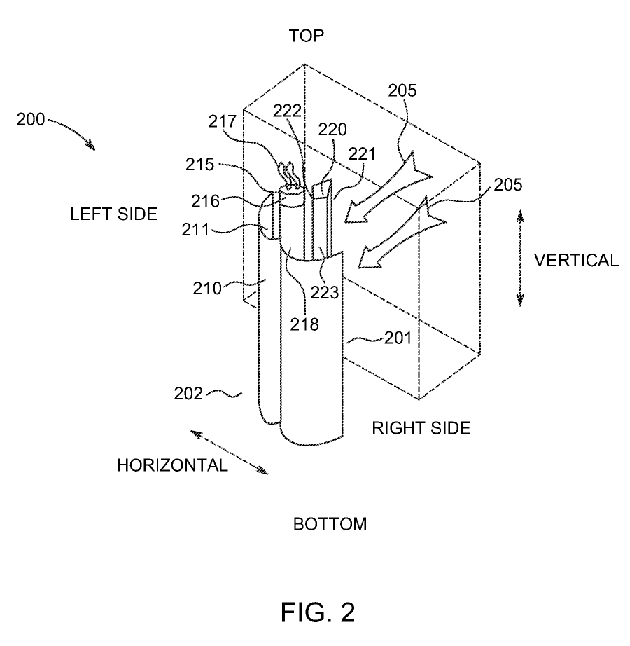

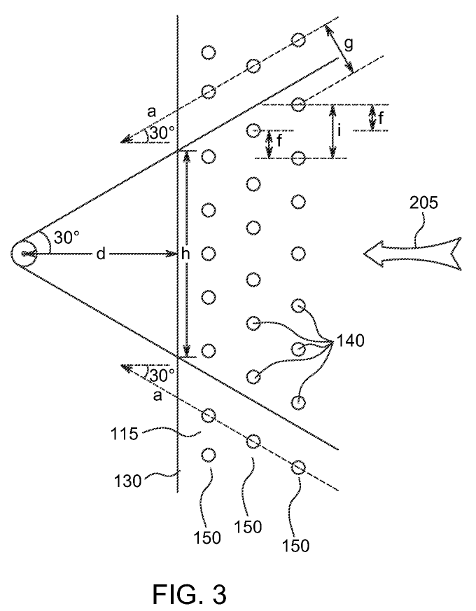

In order to form a fluid passage and to maximize exposure of the finned tubing 110 to flowing air, cooling coils are commonly arranged in a serpentine configuration such as the one shown in FIG. 1. As shown, the coil 105 comprises a serpentine portion 135 which comprises a plurality of straight portions 140 which are parallel to one another and spaced apart. The straight portions are arranged in horizontal rows along a horizontal axis perpendicular to the air flow direction (shown in FIG. 2) and which are offset so as to form a quincunx pattern when viewed from a top or bottom cross sectional view, as shown in FIG. 3. FIG. 3 shows a top cross sectional view of a portion of the coil 105 taken along line A-A. Note that the number of straight portions 140 in FIG. 1 and FIG. 3 do not exactly correspond. Indeed the Figures are meant to illustrate possible coil arrangements and have been simplified for ease of comprehension; the skilled person will appreciate that coil tubing configurations and fins spacing can vary.

In this context the terms horizontal and vertical, as well as top and bottom and left and right are meant as relative references and provide a convenient way to describe positions of things relative to one another. However, it will be appreciated that the various parts of the climate control system and its various parts can be oriented differently. For example, the system could be rotated such that the horizontal plane and a vertical plane become vertical and horizontal, respectively. As such these terms are not meant to be construed as absolute constraints on the system. Likewise the terms front and rear may be used in relation with UV decontamination systems to mean the side made for facing airflow (upstream side when so installed) and the opposite side (downstream side if so installed). However, these are relative term designating their relative positions regardless of whether the system is installed in an airflow or not.

The climate control system of this example comprises an ultraviolet decontamination unit 200 which irradiates the coil 105 with ultraviolet (UV) radiation to kill and prevent growth of mold fungus and other organic contaminants. The decontamination unit 200 is mounted in the climate control system and can be mounted downstream from the coil 105 and is oriented towards the coil volume 115 to emit UV radiation towards the coil 105. Advantageously, there is typically more room on the downstream sides of coils in climate control systems to mount the decontamination unit 200 than on the upstream side. However, on the downstream side, the decontamination unit is exposed to cooling air and mist as mentioned.

The decontamination unit 200 of this example has first and second opposed sides 201, 202, which as mounted here are an upstream side and a downstream side, and a body defined between the first and second opposed sides 201, 202. The decontamination unit comprises a UV lamp 215, a reflector 210 mounted on one side of the UV lamp towards the first side 201 and a deflector 220 mounted on another side of the UV lamp towards the second side 202. In this example, the reflector 210 and the deflector 220 are mounted on opposed sides, though this is not necessarily so. Parts of the body are omitted from the illustration in the interest of making other parts visible. In particular, a frame linking together the reflector 210, UV lamp 215 and deflector 220 is not shown.

The UV lamp 215 is a lamp for emitting UV radiation. The UV lamp is configured to hold a UV light source such as a UV emitting tube or bulb. To this end, the UV lamp 215 comprise a UV lamp mount 216 onto which is or can be mounted a UV light source. In the example shown, the UV lamp mount 216 is a UV tube mount comprising two bases, one for each side of a UV tube (only one of which is visible in FIG. 2) which receive the electrodes of a UV tube and holds it in place.

The UV light source is accommodated by the UV lamp 215 in a lamp volume 218 defined in the UV lamp 215. When an appropriate UV light source is provided in the UV lamp 215, the lamp volume 218 is filled by the UV light source. However, UV light sources such as UV tubes are typically replaceable parts that may or may not be provided with the UV lamp 215. In the example shown in FIG. 2, a UV light source is provided in the lamp volume 218. In alternate embodiments, a UV lamp may be provided with a permanently mounted UV light source permanently filling the lamp volume 218.

The UV lamp 215 also comprises an electrical power source 217 for powering the UV light source. In the example shown the electrical power source 217 comprises a pair of wires for providing electricity from another source (e.g. a wall outlet) to the UV light source. In this example, the electrical power source also comprises a ballast, although this is not shown in FIG. 2.

The reflector 210 is provided in the decontamination unit 200 on a second side of the lamp volume 218 towards the second side 202 of the decontamination unit, which in this example is also downstream from the UV lamp 210. In the example shown here, the UV light source is a UV tube which emits UV radiation in 360 degrees around the tube along its entire length. If it were mounted alone, it would irradiate the coil 105 on one side, but the significant portion of UV rays emitted on the other side would be lost. The reflector 210 has a UV light reflective surface 211 which is oriented towards the upstream direction, particularly towards the first side 201 of the decontamination unit 200 and more particularly towards the coil 105 and even more particularly towards the downstream side 130 of the coil 105, meaning that it is positioned to reflect UV light from the UV source in that direction.

The deflector 220 is provided in the UV decontamination unit 200 on a second side of the lamp volume 218. In particular the deflector is provided on a first side of the lamp volume 218 towards the first side 201 of the UV decontamination unit 200 in a direction upstream from the expected airflow 205. The deflector 220 creates a deflecting obstruction to airflow travelling from the first side 201 towards the second side 202 of the UV decontamination unit 200 in front of the lamp volume 218. The deflector 220 curves the airflow away from the lamp volume 218 such that the (often cold and misty) airflow does not hit a UV light source located in the lamp volume.

As such the deflector 220 serves as an anti-fouling shield for any UV light source in the lamp volume 218 preventing the accumulation of mist against the UV light source. The inventors have recognized that moisture, bio-aerosols, dust and the like that are carried in the airflow downstream of the cooling coil 105 can impact UV light sources and dry/accumulate residue thereon causing a fouling film to accumulate on the UV light source. This has a detrimental effect as it can reduce the UV output of the UV light source, and moreover requires maintenance to clean the UV light source. Since UV decontamination is sometimes intended to reduce the maintenance requirement of cleaning the cooling coil 105, this is a self-defeating situation at least in this respect.

Perhaps even more importantly, the inventors have recognized that another danger lies in placing UV lamps downstream from the coil. In particular, the cold air flowing over the UV light source cools it down, which may very significantly impact lamp performance and UV output. As a result, the UV lamp becomes less effective at decontaminating the coil 105 leading to potential health risks.

Thus the deflector provides an important advantage significantly improving the performance of the UV decontamination unit 200 making it effective even when mounted on the downstream side of the coil 105.

The deflector has a first side 221 facing the first side 201 of the UV decontamination unit 200. The first side 221 of the deflector 220 has a geometry deflecting airflow laterally (in this example horizontally left and right) from the lamp volume 218. To this end, the first side 221 of the deflector 220 has a face angled transversally to the direction of airflow (or more specifically, the direction of a heading from the first side 201 to the second side 202 of the UV decontamination unit 200) to impart a change of direction of the airflow to curve the flowing air away from the lamp volume 218. In particular, the first side here 221 has a the profile of a top of an isosceles triangle, parting the airflow around both left and right sides of the lamp volume 218.

In alternate embodiments the first side 221 could have a geometry deflecting airflow only to one side of the lamp volume 218, for example by having a single angled face.

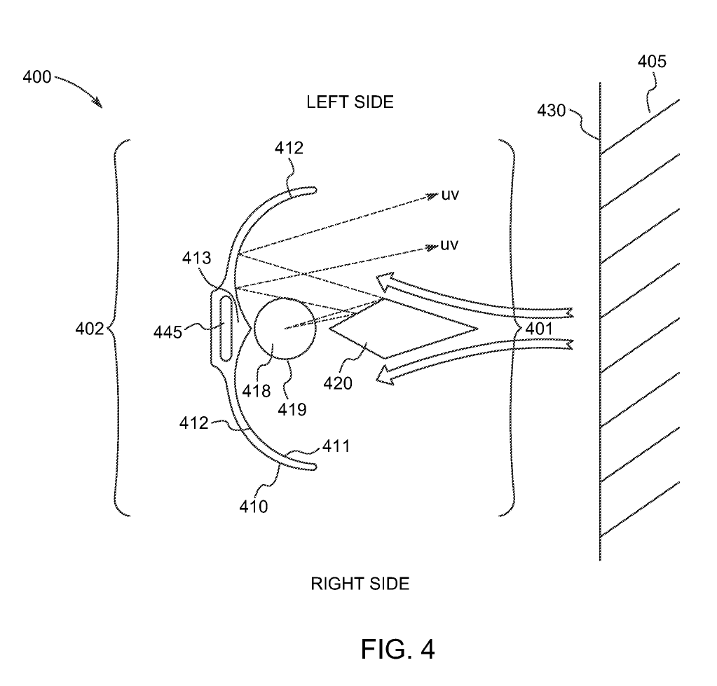

The deflected airflow in this example, and in the example of FIG. 4, is directed in part towards the UV reflective surface 211. As a result some of the mist and other particles carried by the flowing air may land upon the reflector 210. However, unlike the UV light source, the reflector 210 is not a source of heat. Although it is irradiated by the UV light source, a large portion of the energy is reflected away and the reflector 210 is typically much cooler than the UV light source and consequently less prone to fouling as water droplets may bead off. Moreover, cooling of the reflector by the airflow is not necessarily problematic. That being said water droplets on the reflector 210 may attenuate somewhat the UV light reflected back by the reflector 210 and some fouling may occur, which also may attenuate the UV light.

In this particular example, the second side 222 of the deflector 220 has a UV reflective surface 223 which is oriented towards the downstream direction, particularly towards the second side 202 of the decontamination unit 200 and more particularly towards the UV reflective surface 211 of the reflector 210, meaning that it is positioned to reflect UV light from the UV source in that direction. UV light emanating from the UV light source towards the deflector 220 is therefore not merely blocked but is reflected back towards the reflector 210 which may in turn reflect it towards the coil 105. To that end, the second side has a geometry for reflecting UV light irradiated towards it from the lamp volume 218 towards the reflector 210. In particular, in this example the second side 222 has two angled faces oriented towards the UV reflective surface 211 and more particularly towards curved portions thereof.

Advantageously, besides recuperating light that would otherwise be merely blocked, the reflectivity of the deflector 220 may also prevent the deflector 220 from absorbing too much UV light and overheating.

Similarly to the reflector 210, the deflector 220 may be made of extruded aluminum with the same benefits as already described.

FIG. 4 shows a top plan view of a cross section of a decontamination unit 400 according to another particular example of implementation. Like in the previous example, the decontamination unit 400 comprises a UV lamp which in this example is provided with a UV light source 419 in a lamp volume 418, a reflector 410 and a deflector 420. Like in the previous example, the decontamination unit 400 is mounted downstream from a coil 405 and facing a downstream side 430 of the coil 405. The deflector 420 of this example is similar to the deflector 220 of the Example of FIG. 2.

Like in the previous example, the reflector 410 is not merely a flat reflective sheet, but has a geometry that imparts a direction to the UV rays from the UV source that it reflects. A flat sheet would reflect UV light from the UV source at an angle equal and opposite the angle of incidence. As a result some of the UV light would be reflected light away from the coil 105, some of the UV light would be reflected back towards the UV lamp, and much of the UV light, projecting laterally (“horizontally”) on the left and right side would miss both the reflector 410 and the coil 105 altogether. Instead, the reflector has a UV reflective surface 411 that has a curved portion 412 that has a curved profile that concentrates rays received on the UV reflective surface 411 towards the downstream side 130 of the coil 405.

In this example, the reflector 410 is made of extruded aluminum. Aluminum is a well-suited material as it is a good reflector of ultraviolet light in the frequencies used to decontaminate. It is also usefully suited for extrusion which allows the creation of a smooth curve profile. That being said with alternate fabrication methods, or even with extrusion, it is possible that the curved portion have an imperfect curve profile composed of joined flat section together approximating a curve.

The curved portion partially surrounds the lamp volume 418. This has the advantage not only of imparting an angle to the reflected UV light that directs it towards the coil 405, but also allows the UV reflective surface 411 to capture and reflect more UV light than a flat reflective surface would.

In this example, the reflector 410 is configured to limit reflection of UV light back towards the UV light source 419. To this end, the UV reflective surface 411 has a geometry that limits reflection of the UV light emanating from the UV source back towards the lamp volume 418 and the UV light source 419. In particular here, the UV reflective surface 411 has a projection 413 extending towards the lamp volume 418 to a peak. The projection 413 avoids the presence of a reflective surface area downstream of the lamp volume 418 that is normal to the straight path of the UV light from the lamp volume 418 and that would therefor reflect back towards the lamp volume 418. Instead, the projection 413 is shaped to reflect UV light emanating from the lamp volume 418 to the projection 413 towards another portion of the UV reflective surface 411. Although due to certain factors such as diffusion, imperfections in the profile of the curved portion 412, or the presence of areas of the curved profile that are tangentially normal to the path of UV light from the lamp volume 418, some UV light may still be reflected from the UV reflective surface 411 back towards the lamp volume 418 and UV light source 419, the projection 413 avoids a large amount of such reflection that would otherwise occur and may also be used to direct UV light in a particular direction.

In alternate embodiments, the reflector 410 may be configured to limit the reflection of UV light back towards the lamp volume 418 and UV light source 419 by other mechanisms, such as by having an opaque area or gap where the UV reflective surface 411 would otherwise reflect UV light back to the lamp volume 418.

In this example, the reflector 410, which is made of extruded aluminum, has an opening 445 running its vertical length. The opening 445 has the manufacturing advantage of saving on material, offering a mean for screw assembly, and also provides the advantage of a lighter product.

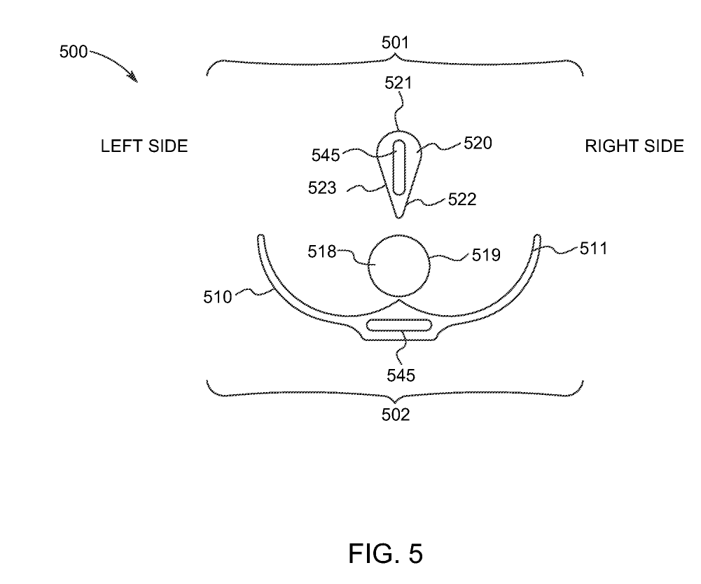

FIG. 5 shows a top plan cross section of another variant of a UV decontamination unit 500. Like in the previous example, the decontamination unit 500 comprises a UV lamp which in this example is provided with a UV light source 519 in a lamp volume 518, a reflector 510 and a deflector 415. Like in the previous example, the decontamination unit 500 is mounted downstream from a coil and facing a downstream side of the coil.

The deflector 520 of this example varies from the previous example in geometry. Like in the example of FIG. 4, the deflector 520 has a first side 521 facing a first side 501 of the UV decontamination unit 500. The first side 521 of the deflector 520 also has a geometry deflecting airflow laterally (in this example horizontally left and right) from the lamp volume 518. But instead of having two angled faces, the first side 521 is curved, and particularly in this case has a semi-circular cross-section and a semi-cylindrical surface.

In this particular example, the second side 522 of the deflector 520 also has a UV reflective surface 523 which is oriented towards the downstream direction, particularly towards the second side 502 of the decontamination unit 500 and more particularly towards a UV reflective surface 511 of the reflector 510. To that end, the second side has a geometry for reflecting UV light irradiated towards it from the lamp volume 518 towards the reflector 510 and in particular has two angled faces oriented towards the UV reflective surface 511. In this example, however, the angled faces have a smaller angle to one another and a longer profile.

In this example, openings 545 are provided in the reflector 510, as in the example of FIG. 4, but also in the deflector 520 for similar reasons.

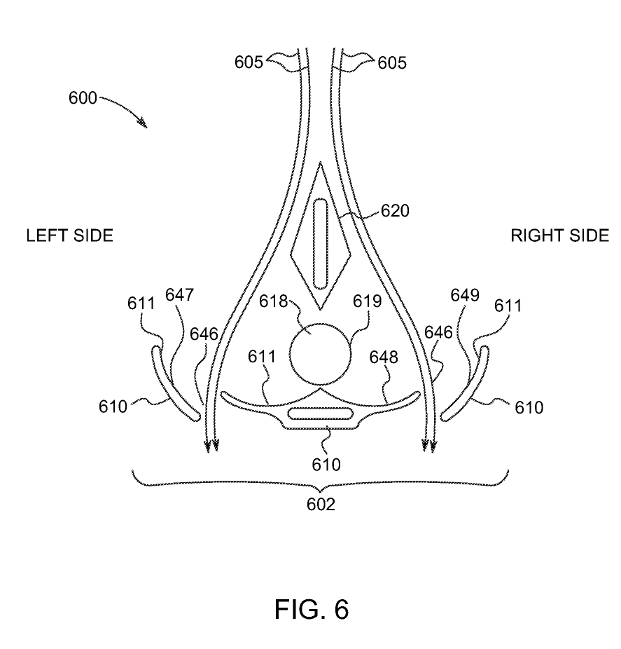

FIG. 6 shows a top plan cross sectional view of yet another example of UV decontamination unit 600. Like in the previous example, the decontamination unit 600 comprises a UV lamp which in this example is provided with a UV light source 619 in a lamp volume 618, a reflector 610 and a deflector 620. Like in the previous example, the decontamination unit 600 is mounted downstream from a coil and facing a downstream side of the coil.

In this particular case, however, the reflector 610 has openings 646 in the left and right sides of the UV reflective surface 611. In some cases, the openings 646 may be finite in vertical length, like a slot that does not go up the entire vertical length of the reflector 610. In such a case the UV reflective surface 611 may be a non-continuous plane having holes in it. In this particular case, however, the openings 646 run the entire vertical length of the reflector 610 essentially separating the UV reflective surface into a left, center, and right portion 647, 648, and 649. The disjoined UV reflective surface 611 thus has air channels (openings 646) allowing the airflow to pass through the reflector 610. As shown the airflow 605 diverted by the deflector 620 towards the reflector 610 can now pass through the reflector 610.

This embodiment allows a reduction of the drag caused by the UV decontamination unit 600 in the climate control system. However, a particular advantage of this configuration is that droplets and other contaminants carried by the airflow can bead off the UV reflective surface 611 through opening 646, thereby keeping the UV reflective surface 611 cleaner and therefore more reflective.

Although openings 646 can merely be provided in the form of gaps in a particular curve profile, in order to maximize catchment of UV radiation, the reflector 610 has a geometry that ensures complete coverage of the second side of the UV decontamination unit 600 which maximizes UV utilization and minimizes downstream leakage of potentially harmful UV light. To that end, the left and right portions 647, 649 of the disjoined UV reflective are offset, in this case rearwardly (downstream) towards the second side 602 of the UV decontamination unit 600. The left and right portions 647 and 649 may also have a modified curvature vis-à-vis the center portion 648 to widen the curvature to account for a greater distance from a central or focus point such as the center of the lamp volume 618 or UV light source 619.

It will be noted that the deflectors of the above example block UV light radiating directly from the UV light source towards the coil 105 along the shortest path (normal to the downstream side 130 of the coil volume 105 and/or parallel to airflow and/or direction from the second to the first side of the UV decontamination unit) albeit that UV radiation is redirected towards elsewhere on the coil 105. However, the inventors determined that the straight-on UV radiation is not actually the most effective for decontaminating a cooling coil and that UV rays with a certain angle are more effective.

Returning to FIG. 3, we see that the straight portions 140 are spaced apart along lateral rows 150 that are transversal to (and in this example perpendicular to) the airflow direction 205. The straight portions 140 in each row 150 extend in a plane that is transversal (and in this example perpendicular to) the airflow direction 205 and in this example are parallel to the downstream side 130 of the coil volume 115. Now in order to maximize exposure to the airflow and heat transfer, each row is offset laterally from the adjacent one by an offset f. This offset causes the straight portions 140 to align along diagonal axes a. The diagonals a are spaced apart by a gap g. The gap g and the diagonals a necessarily have the same angle. In this particular example the offset f is half the lateral distance i between straight portions, as a result of which the straight portions are arranged in a quincunx pattern.

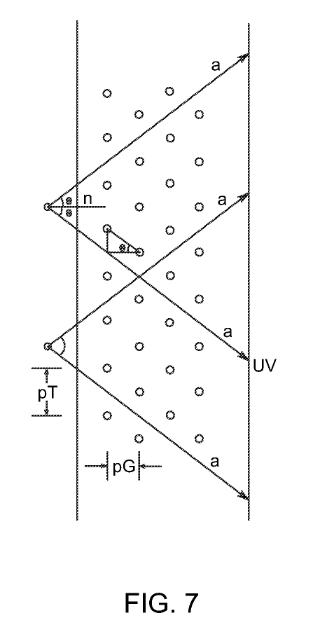

Turning now to FIG. 7, the angle of the diagonals a can be computed based on the lateral distance pT between straight portions 140 and the longitudinal distance pG between them. In particular, we find the angle θ between the longitudinal line n (normal to the downstream side 130 of the coil volume 105 and/or parallel to the airflow direction and/or extending from the second to the first side of the UV decontamination unit) and the diagonal a. This can be found as follows:

$$θ = arctan ( \frac{pT}{2 pH} ) = 30 ° .$$

Now the inventors have discovered that in order to maximize decontamination effectiveness, UV light rays should be directed down the gaps g between the diagonals, and therefore at the angle of the gap stretching between the diagonal rows a of straight portions 140. Indeed, with direct straight-on UV irradiation, the first lateral rows (two rows in the case of a quincunx arrangement) receive the full force of the UV irradiation on one of their sides while casting shadows on the on the rest of the rows. However, if the UV light rays can be directed at an angle θ to the longitudinal, then the UV light will propagate down the diagonal gaps and permeate through substantially the entire coil volume 115 to decontaminate substantially the entire coil fins and tubes 105 or the straight portions 140 thereof.

The inventors have discovered that the UV light from the UV light source can be imparted a prevailing angle θ that is equivalent to the diagonal angle to provide better UV penetration of the coil fins 105 and better decontamination.

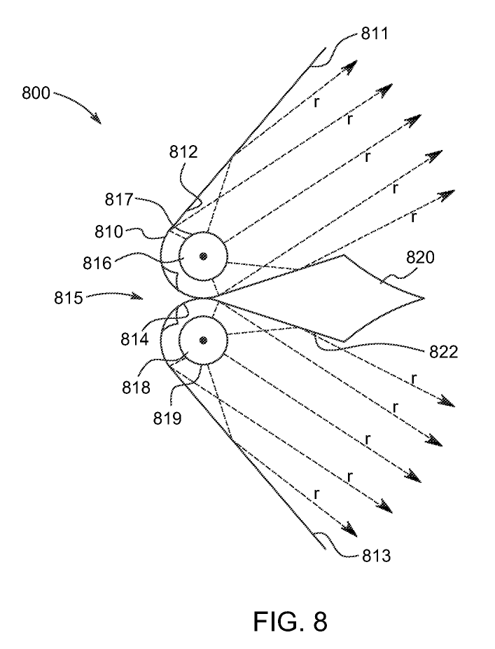

FIG. 8 shows a top plan cross sectional view of another UV decontamination unit 800 according to another example. In this example, UV decontamination unit 800 has a reflector 810, a deflector 820, and a UV lamp 815, however in this example the UV lamp is a UV lamp for two UV light sources (in this example UV tubes) and has two lamp volumes namely first lamp volume 816, and second lamp volume 818 accommodating in this case a UV light source each, namely first UV light source 817 and second UV light source 819. The UV lamp also has correspondingly two lamp mounts (not shown) and the power source is for powering the two UV light sources 817, 819.

Now in this particular example the reflector 810 has two UV reflective surface portions, namely a first UV reflective surface portion 811 and a second UV reflective surface portion 813. Each of the UV reflective surface portions 811, 813 have respective curved portions 812, 814. In particular the curved portion is profiled to impart a prevailing angle to the rays. In this particular case, the curved portion has an at least partially parabolic profile with the respective lamp volumes 816, 818 (or UV light sources 817, 819) at the focus of the parabola. Preferably, the center of the respective lamp volumes 816, 818 (or UV light sources 817, 819) is at the focus. In the present example the UV reflective surface portions 811, 813 comprise a generally parabolic profile that tapers into straight segments.

Each UV reflective surface portions 811, 813 reflects UV light rays stemming from the focus at a prevailing angle whereby individual rays r generally travel in the same (generally parallel) direction, albeit possibly spaced apart, as shown in FIG. 8. The reflective surface portions 811, 813 are oriented such as to be oriented towards the prevailing angle, such that the UV rays reflected in this manner adopt the prevailing angle which may be defined as an angle with respect to the longitudinal described above. Thus the UV reflective surface portions 811, 813 may be profiled to impart a prevailing angle to the rays so as to irradiate the coil volume at an angle non-normal to a downstream face of the coil volume

It should be noted that although this example comprises two UV light sources 116, 818 and two UV reflective surface portions 811, 813, similar geometry may be used in a single UV light source and single UV reflective surface portion to impart a prevailing angle and direct UV light as described.

In the present example, the deflector 820 is adjacent with the reflector 810 such that they touch. As such, the UV reflective surface portions 811, 813 may be considered to each extend into the second (downstream) side 822 of the deflector 820. Thus the second side 822 of the deflector 820 may have faces forming extensions of and collaborating with the UV reflective surface portions 811, 813. These faces may have a curvature for forming part of a curved surface, such a parabola.

Returning to FIG. 3, we can compute the distance d at which a UV light source must be placed from the downstream side 130 in order to achieve light propagation at an angle θ across a lateral width h of the coil 105, or conversely, how wide a lateral width h of the coil 105 will be exposed if the UV decontamination system is mounted at a distance d. In the example of FIG. 7 we computed the angle of the diagonal rows (and gaps therebetween) of straight portions 140 to be 30°. In one example, we use the UV decontamination system 800 of FIG. 8, with the UV reflective surface portions 811, 813 oriented such that the rays r are 60° apart on either side, or each 30° from a longitudinal center line. If a mounting point for the UV decontamination system 800 exists at distance d from the downstream side 130 of the coil volume 115, then the width separating the central rays of both sides of the UV decontamination system 800 will be of h where

h=2d tan θ.

Thus with 30° as angle, and at 19″ away from the coil volume 115, we get a width of 20.8″ and at 8″ away, we get a width of 9.2″.

As mentioned, the fins in this example may be made of aluminum. The aluminum fins have the added advantage that they reflect UV light, leading to less loss as the UV light travels across the coil volume. It will be noted that the UV decontamination unit 200 is mounted such that the UV rays are not blocked by the fins, and in particular it is angled such that the prevailing angle(s) provided to the UV light is in the plane of the fins such that the UV light travels diagonally between the surfaces of the fins (unblocked by the fin surfaces) rather than being blocked by the fins. To this end, the UV decontamination unit 200 may be installed lengthwise parallel to the straight portions 140. For example in one example where the UV light source is a UV tube, the UV tube is placed in the same direction as the straight portions 140.

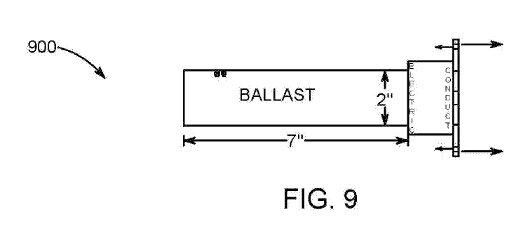

FIG. 9 illustrates a ballast 900 for a UV lamp, which may be part of the electric power source. The ballast 900 may be conveniently located in a UV decontamination system so as to be out of the way and protected from UV radiation. In one example, the ballast may be incorporated into a reflector.

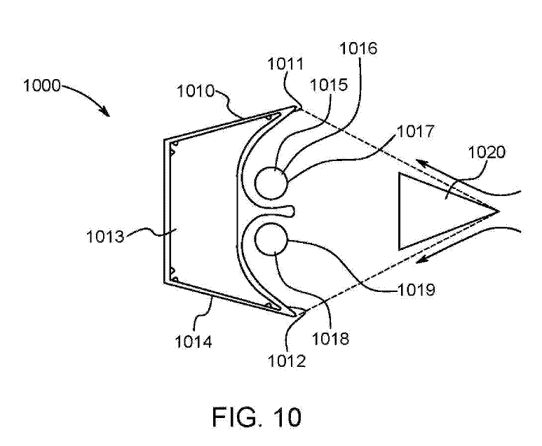

FIG. 10 illustrate another top plan cross sectional view of another UV decontamination unit 1000 according to another example. In this example, UV decontamination unit 1000 has a reflector 1010, a deflector 1020, and a UV lamp 1015, however in this example the UV lamp is a UV lamp for two UV light sources (in this example UV tubes) and has two lamp volumes namely first lamp volume 1016, and second lamp volume 1018 accommodating in this case a UV light source each, namely first UV light source 1017 and second UV light source 1019. The UV lamp also has correspondingly two lamp mounts (not shown) and the power source is for powering the two UV light sources 1017, 1019. Similarly to the example of FIG. 8, the reflector 1020 has two UV reflective surface portions 1011, 1012.

The reflector 1010 has a frame 1014 which in this example is extruded aluminum and the entire reflector 1010 is a single piece. The frame 1014 is part of the body of the UV detection system 1000 and may comprise mounting portions for mounting the system in a climate control system. Moreover, the frame comprises a cavity 1013 which is similar to opening 545 but in this case is much bigger. In this example, the cavity 1013 defines a ballast volume suitable for accommodating ballast 900 and comprises mounting hardware for affixing the ballast 900 in place such as screw holes or the like.

Although in the examples provided the UV decontamination systems were always described in relation to a downstream mounting location relative to the cooling coil, it is to be understood that these systems may, if so desired be mounted elsewhere such as on the upstream side of the coil and oriented towards the coil.

Herein description has been provided with reliance on top plan cross-sectional views. In such description planar language may have been used, however it should be understood that the devices described may extend vertically in three dimensions. Descriptions may thus apply over a vertical range such that a semi-circular shape, for example becomes a cylindrical face. This does not preclude, however, the possibility of applying variations (e.g. in angles, curvature profile, etc. . . . ) as the vertical elevation varies.

Although in the description herein common air has been assumed to be the fluid in external contact with the cooling coil, there may be other embodiments where other types of fluids are used.

UV decontamination systems have been described as being in a climate control system. Climate control system typically include an at least partially enclosed air circulation system sometimes enclosed by ducts. However, there may be systems that use forced air without, in some parts ducts. A UV decontamination system may be considered to be in a climate control system if it is mounted within its air ducts but also if it is mounted within the force flow of air in proximity to a coil or other component to decontaminate.

Although the described examples have been for decontaminating ducts, the systems described may have applications elsewhere, such as for other components or a climate control system or in other places.

Although various embodiments have been illustrated, this was for the purpose of describing, but not limiting, the present invention. Various possible modifications and different configurations will become apparent to those skilled in the art and are within the scope of the present invention, which is defined more particularly by the attached claims.

What is claimed is:

1. A cooling unit for a climate control system comprising:
a. a finned cooling coil comprising a fluid conductor occupying a coil volume having an upstream side and a downstream side, the finned cooling coil including a finned fluid passage between the upstream side and the downstream side allowing flow of air through the finned cooling coil between the upstream side and the downstream side such that, in use, cooled air having been cooled by the cooling coil flows downstream away from the coil volume;
b. an ultraviolet irradiation unit mounted within a path of airflow on the downstream side of the coil volume for irradiating the finned cooling coil with ultraviolet light for decontaminating the finned cooling coil, the ultraviolet irradiation unit comprising
i. an ultraviolet lamp having an electrical power source for powering an ultraviolet light source and a lamp mount for accommodating the ultraviolet light source in a lamp volume;
ii. a reflector mounted downstream from the lamp volume and having a ultraviolet light-reflective upstream surface oriented generally upstream to reflect ultraviolet radiation from the ultraviolet light source towards the finned cooling coil;
iii. a deflector mounted within the path of airflow upstream from the lamp volume creating an obstruction deflecting air flow around the lamp volume, the deflector having an upstream-facing side having a geometry imparting in use a change of the direction of the cooled air downstream past the lamp volume and around both sides thereof.

2. The cooling unit of claim 1, wherein the upstream-facing side comprises at least one face angled transversally to the direction of air flow at an inclination at an angle sufficient to provide an airflow angle change to curve the air flow away from the lamp volume.

3. The cooling unit of any of claims 1-2, wherein the deflector has a light-source facing side comprising a ultraviolet light-reflective downstream surface oriented to reflect ultraviolet light from the light source towards the ultraviolet light-reflective upstream surface.

4. The cooling unit of claim 3, wherein the ultraviolet light-reflective downstream side forms an extension of the ultraviolet light-reflective upstream surface.

5. The cooling unit of claim 4, wherein the ultraviolet light-reflective downstream side forms part of a curved surface along with the ultraviolet light-reflective upstream surface.

6. The cooling unit of any of claims 1-5, wherein the first ultraviolet light-reflective surface comprises a curved portion to concentrate towards the finned cooling coil the reflection of rays emitted by the ultraviolet light source towards curved portion.

7. The cooling unit of claim 6, wherein the curved portion is profiled to impart a prevailing angle to the rays so as to irradiate the coil volume at an angle non-normal to a downstream face of the coil volume.

8. The cooling unit of claim 7, wherein the downstream face of the coil volume is a planar face having a normal, the at least one curved portion is profiled to impart a prevailing angle of substantially 30 degrees to the normal of the downstream face of the coil volume.

9. The cooling unit of claim 7, wherein the finned cooling coil comprises a serpentine tubular portion comprising a plurality of straight segments within the coil volume that are parallel to one another and spaced apart, and wherein the finned fluid passage comprises a plurality of straight paths across the volume at a path angle that is non-normal to the face of the coil volume, and wherein the at least one curved portion is profiled to impart a prevailing angle that is substantially parallel to the path angle.

10. The cooling unit of claim 9, wherein the plurality of straight segments are spaced apart into a plurality of lateral rows that are laterally offset from one another so as to form diagonal rows of straight portions separated by gaps, wherein the at least one curved portion is profiled to aim reflected rays through gaps between the diagonal rows.

11. The cooling unit of any of claims 6-10, wherein the curved portion comprises a parabolic portion.

12. The cooling unit of any of claims 7-11, wherein the ultra-violet light reflective surface is a first ultra-violet light reflective surface portion and the curved portion is a first curved portion, the prevailing angle is a first prevailing angle and the rays are first rays, wherein the reflector further comprises a second ultra-violet light reflective surface portion having a second curved portion profiled to impart a second prevailing angle to second rays emitted by the ultraviolet light source towards the second curved portion so as to irradiate the coil volume at an angle non-normal to a downstream face of the coil volume, wherein the second prevailing angle is opposite the first prevailing angle.

13. The cooling unit of claim 12, wherein the ultraviolet light source is a first ultraviolet light source, the lamp volume is a first lamp volume, the lamp mount is a first lamp mount is a first lamp mount, and the power source is for powering two ultraviolet light sources, wherein the ultraviolet lamp has a second lamp mount for accommodating a second ultraviolet light source in a second lamp volume, wherein the first curved portion curves partially around the first lamp volume and the second curved portion curves partially around the second lamp volume.

14. The cooling unit of any of claims 1-12, wherein the ultraviolet light-reflective upstream surface is profiled to prevent reflection of ultraviolet light from the light source back towards the light source.

15. The cooling unit of claim 14, wherein the ultraviolet light-reflective upstream surface comprises a projection extending towards the lamp volume to a peak which is shaped to reflect ultraviolet light emanating from the light source towards another portion of the light-reflective upstream surface.

16. The cooling unit of claim 12, wherein the ultraviolet light source is a first ultraviolet light source, the lamp volume is a first lamp volume, the lamp mount is a first lamp mount is a first lamp mount, and the power source is for powering two ultraviolet light sources, wherein the projection is a first projection extending towards the first lamp volume to a first peak, wherein the ultraviolet light-reflective upstream surface comprises a second projection extending towards the second lamp volume to a second peak which is shaped to reflect ultraviolet light emanating from the second light source towards another portion of the light-reflective upstream surface.

17. An ultraviolet decontamination unit for decontaminating a finned cooling coil in a climate control system comprising:
a. a first side for facing the finned cooling coil;
b. a second side opposite the first side;
c. a body defined between the front and rear side along a front-rear axis and mountable in the climate control system within a path of airflow of the climate control system in proximity to the finned cooling coil with an orientation wherein the front side faces the finned cooling coil and the path of airflow is directed generally towards the front side of the body such that, in use, cooled air having been cooled by the cooling coil flows downstream towards the body from an upstream side of the body to a downstream side of the body, the body comprising:
i. an ultraviolet lamp located between the front and rear side for generating ultraviolet light to irradiate the finned cooling coil having an electrical power source for powering an ultraviolet light source and a lamp volume for accommodating the ultraviolet light source;
ii. a reflector located towards the rear from the lamp volume and having a first ultraviolet light-reflective front face oriented towards the lamp volume to reflect ultraviolet radiation from the ultraviolet light source towards the front;
iii. a deflector located towards the front from the lamp volume creating an obstruction and having a front-facing side having a geometry imparting in use a change of the direction of the cooled air deflecting the cooled air downstream past the lamp volume and around both sides thereof towards the downstream side of the body to limit cooling of the ultraviolet light source.

18. The cooling unit of claim 17, wherein the front-facing side comprises at least one face angled transversally to the front-rear axis at an inclination at an angle sufficient to provide an airflow angle change to curve the air flow away from the lamp volume.

19. The cooling unit of any of claims 17-18, wherein the deflector has a light-source facing side comprising a ultraviolet light-reflective downstream surface oriented to reflect ultraviolet light from the light source towards the ultraviolet light-reflective upstream surface.

20. The cooling unit of claim 19, wherein the ultraviolet light-reflective downstream surface forms an extension of the ultraviolet light-reflective upstream surface.

21. The cooling unit of claim 22, wherein the ultraviolet light-reflective rear side forms part of a curved surface along with the ultraviolet light-reflective upstream surface.

22. The cooling unit of any of claims 17-21, wherein the first ultraviolet light-reflective surface comprises a curved portion to concentrate towards frontward direction the reflection of rays emitted by the ultraviolet light source towards curved portion.

23. The cooling unit of claim 22, wherein the curved portion is profiled to impart a prevailing angle to the rays so as to irradiate the coil volume at an angle to the front-rear axis.

24. The cooling unit of claim 23, wherein the at least one curved portion is profiled to impart a prevailing angle of substantially 30 degrees to the front-rear axis.

25. The cooling unit of claim 23, wherein the at least one curved portion is profiled to impart a prevailing angle that is substantially parallel to and angled path through the finned cooling coil.

26. The cooling unit of claim 25, wherein the at least one curved portion is profiled to aim reflected rays through gaps between diagonal rows of tubular sections of the finned cooling coil.

27. The cooling unit of any of claims 22-26, wherein the curved portion comprises a parabolic portion.

28. The cooling unit of any of claims 23-27, wherein the ultra-violet light reflective surface is a first ultra-violet light reflective surface portion and the curved portion is a first curved portion, the prevailing angle is a first prevailing angle and the rays are first rays, wherein the reflector further comprises a second ultra-violet light reflective surface portion having a second curved portion profiled to impart a second prevailing angle to second rays emitted by the ultraviolet light source towards the second curved portion so as to irradiate frontwards at an angle to the front-rear axis, wherein the second prevailing angle is opposite the first prevailing angle.

29. The cooling unit of claim 28, wherein the ultraviolet light source is a first ultraviolet light source, the lamp volume is a first lamp volume, the lamp mount is a first lamp mount is a first lamp mount, and the power source is for powering two ultraviolet light sources, wherein the ultraviolet lamp has a second lamp mount for accommodating a second ultraviolet light source in a second lamp volume, wherein the first curved portion curves partially around the first lamp volume and the second curved portion curves partially around the second lamp volume.

30. The cooling unit of any of claims 17-28, wherein the ultraviolet light-reflective upstream surface is profiled to prevent reflection of ultraviolet light from the light source back towards the light source.

31. The cooling unit of claim 30, wherein the ultraviolet light-reflective upstream surface comprises a projection extending towards the lamp volume to a peak which is shaped to reflect ultraviolet light emanating from the light source towards another portion of the light-reflective upstream surface.

32. The cooling unit of claim 28, wherein the ultraviolet light source is a first ultraviolet light source, the lamp volume is a first lamp volume, the lamp mount is a first lamp mount is a first lamp mount, and the power source is for powering two ultraviolet light sources, wherein the projection is a first projection extending towards the first lamp volume to a first peak, wherein the ultraviolet light-reflective upstream surface comprises a second projection extending towards the second lamp volume to a second peak which is shaped to reflect ultraviolet light emanating from the second light source towards another portion of the light-reflective upstream surface.
